(19) United States
(12) Patent Application Publication
(10) Pub. No.: US 2026/0132367 A1
(43) Pub. Date: May 14, 2026

(54) ISOLATION KIT

(71) Applicant: EXOCUBE BIOTECHNOLOGY LIMITED, South Yorkshire (GB)

(72) Inventor: Kemal Tunc TIRYAKI, Istanbul (TR)

(21) Appl. No.: 18/697,508

(22) PCT Filed: Jan. 18, 2023
(86) PCT No.: PCT/TR2023/050037

§ 371 Date: Apr. 1, 2024

(51) Int. Cl. C12M 1/26; B01L 3/00; G01N 1/40

(52) U.S. Cl. CPC C12M 33/14; B01L 3/502761; G01N 1/4005; B01L 2200/0647; B01L 2400/0487; G01N 2001/4016

(57) ABSTRACT

A portable and combined isolation kit is provided. The isolation kit enables obtaining activated exosome molecules, nanoparticles or protein molecules in certain sizes from various sources including cell culture, biological material, plant, and blood plasma

### CROSS REFERENCE TO THE RELATED APPLICATIONS

This application is the national phase entry of International Application No. PCT/TR2023/050037, filed on Jan. 18, 2023, the entire contents of which are incorporated herein by reference.

### TECHNICAL FIELD

The present invention relates to a portable and combined product that will enable obtaining activated exosome molecules, nanoparticles or protein molecules in certain sizes from various sources including cell culture, biological material, plant, blood plasma.

In particular, the present invention relates to a single or reusable alternative product that uses dead-end filtration and tangential-flow filtration system, which can be used with a single medical device without disturbing the closed system conditions to obtain glass beads for activation, by concentrating exosome molecules, nanoparticle or protein molecules in certain size ranges so as to obtain activated and filtered exosome molecules, nanoparticles or protein molecules in certain size ranges.

### BACKGROUND

Developing therapeutic strategies to repair and regenerate damaged tissues and restore their normal functions has become the most important goal of regenerative medicine with the increasing incidence of degenerative and traumatic diseases due to the aging of the human population. Developing innovative technologies that can regulate cellular functions and activate innate regeneration potentials based on materials science and engineering is a rising trend and promising future within the framework of this target.

Multicellular organisms secrete chemical messages that can travel long distances as they need to communicate for billions of cells to perform their functions simultaneously. This communication that cells establish through the molecules they secrete can be classified as local and long distance. Local communication is the communication of cells with other cells in their environment and when it comes to local regulators such as neurotransmitters, hormones and extracellular vesicles are actively involved in long-distance communication. Extracellular vesicles are lipid-layer nanovesicles that secrete their rich biological contents consisting of microRNA, mRNA, DNA and proteins into the extracellular environment and also perform critical functions such as intercellular signal transduction and genetic material transfer. Extracellular vesicles are divided into three groups as apoptotic bodies, microvesicles and exosomes according to their biological formation, size and cellular origin.

During the recent years, the potential of exosome-based therapies has attracted great interest in regenerative medicine. When it was first discovered, although the unwanted material by the cell was called cell waste, it has been proven that exosomes have a large positive role in the immune system, suppression of inflammation, increase of mitochondrial activity and many metabolic events as a result of researches. Exosomes are vesicles between 20-130 nm in size, produced in the plasma by all cells and secreted out of the cell. The nanoscale size of exosomes prevents them from being phagocytosed by mononuclear macrophages and also allows them to easily pass through various biological barriers such as the blood vessel wall and the blood-brain barrier. Exosomes accelerate tissue repair by increasing cell proliferation as well as skin repair, skin rejuvenation, thus reducing inflammation. They also have therapeutic applications to allow regulation of immunological function in the case of autoimmune suppression and immunosuppression due to their immunosuppressive and immunomodulatory properties. Meanwhile, exosomes have great potential for cell-free regenerative medicine and are promising for new studies to be made.

Although exosomes are released from many cell types, including stem cells, immune cells, dendritic cells, endothelial and epithelial cells, cancer cells, they can also be obtained from various physiological fluids such as serum, cerebral fluid and urine etc. However, the introduction of extracellular vesicles obtained by cell culture into clinical practice has been prevented by the high regulation requirements for ex vivo cell expansion. The use of platelets offers some advantages compared to EVs, especially in terms of safety and regulatory concerns. On one hand, allogeneic platelets can be obtained from whole blood donations as a byproduct of obtaining red blood cells at the clinical level. On the other hand, inhibition of ex vivo cell expansion, being human origin, and lack of growth medium components reduces concerns about contamination or immunological safety compared to the other cell sources. For these reasons, although comparatively little attention has so far been given to the therapeutic use of platelet EVs (pEVs), platelets and their concentrates offer a potential resource for regenerative medicine that overcomes the limitations of other sources of EV.

The latest preclinical studies in regenerative medicine have focused on mesenchymal stem cells (MSCs) for more than a decade because of their differentiation capacity, potent immunomodulatory properties, and ability to be cultured and manipulated properly. In addition to bone marrow, MSCs can be isolated from a variety of adult tissues, including adipose tissue, amniotic fluid, dental pulp, placenta, umbilical cord blood, Wharton gel, and even brain, kidney, liver, lung, spleen, pancreas, and thymus. MSCs can differentiate into different lineages of mesenchymal origin, as well as multiple non-mesenchymal lineages such as such as adipocytes, endothelial cells, cardiomyocytes, chondrocytes and osteoblasts as well as hepatocytes and neuron-like cells. The unique capacity of MSCs to proliferate and differentiate into various cellular phenotypes in vitro offered a great opportunity for their use as therapeutic agents so as to heal necrotic or apoptotic cells of connective tissue. Lately, MSCs have been widely used in clinical trials as a regenerative agent so as to treat various conditions such as osteoarthritis, pulmonary fibrosis, spinal cord injury, myocardial injury, knee cartilage injury, dental pulp regeneration, and organ transplantation. In addition to this, there are some limits to their application of MSCs. Although direct cell transplantation is performed, it is difficult to predict cell survival after cell injection to sustain effects resulting from intercellular interactions and to maintain adequate cellular storage. An increasing number of studies have revealed that the potent therapeutic effects of MSCs are mainly due to their paracrine effects. Extracellular EVs are the principal paracrine effectors of MSCs and play a crucial role in intercellular communication found in various body fluids and cell supernatants. MSC-derived EVs have a wide range of prospective therapeutic applications with advantages over cell therapy, because they have the function of protocells and have lower immunogenicity. Numerous studies have shown that MSC-derived EVs are curative in various diseases including tumor, neurodegenerative diseases, cardiovascular and cerebrovascular diseases, wound repair, etc.

Adipose stem cells (ASCs) isolated from adipose tissues have emerged as a promising therapy for healing multiple tissues such as wound healing, fat graft, skin rejuvenation, cartilage and bone regeneration. ASCs are particularly valuable since they are readily available from the patient due to their abundance in the body and favorable separation properties. ASCs are not only the precursors of adipocytes but also they are multipotent progenitors of various cells, including osteoblasts, chondrocytes, myocytes, epithelial cells and neuronal cells. ASC-derived exosomes carry the specific content of major ASCs, including DNAs, RNAs, lipids, cytokines, and enzymes. ASC-exosomes can protect their cargo intact, are highly stable in serum and blood, thus effectively delivering cargo to target cells. ASC-exosomes are used as vehicles for repair and regeneration of damaged cells and it is thought that they organize the necessary events for tissue regeneration, immune function, tissue homeostasis, and cell fate development. For this reason, although ASC-exosomes are not capable of differentiation, they can mimic the capacity of ASCs for innovative cell-free therapy such as tissue regeneration and repair, injury reduction and inflammation. ASC-exosomes are potentially safer therapeutic agents than ASCs. Furthermore, they have certain advantages over ASCs in production, storage, shelf life, delivery and potentially ready-to-use biological products. ASC-exosomes can avoid the problems associated with cellular therapy, including limited cell survival, immune rejection efficiency, and aging-related genetic instability when compared to ASCs.

PRP, which means platelet-rich plasma, is an autologous derivative of high amounts of human platelets in a small volume of plasma obtained from centrifugation of whole blood, it is used in the treatment of various diseases such as wound healing and connective tissue repair by increasing cell proliferation, and thus the frequency of its application is increasing. Platelets are broadly used in therapeutic applications since they secrete extracellular matrix modulators, together with biomolecules such as growth factors and cytokines, which support cell proliferation, revascularization, and restoration of damaged tissue and activation of mesenchymal stem cells. Although it is primarily used for cosmetic and anti-aging purposes, it has recently started to be used particularly in musculoskeletal system diseases. The results of clinical studies not only showed that PRP treatments are promising, but also revealed that there are some disadvantages and limitations regarding their use that should be taken into consideration. The clinical use of platelet concentrates is actually limited, mainly due to their main disadvantages such as non-standardized separation methods, variability between donors, or lack of reproducibility due to storage conditions. Furthermore, some patients, such as cancer patients or tobacco users, may not be appropriate for such interventions because of their medical history. These disadvantages, combined with the lack of quality control, lead to high heterogeneity of the concentrates obtained. For this reason, platelet-derived extra-cellular vesicles (pEVs) are a promising alternative so as to eliminate the limitations of PRP and other platelet concentrates since it provides a ready-to-use controlled product.

The pEVs, defined as procoagulant particles released by activated platelets, offer an important potential in regenerative medicine treatments. The studies in this field have also increased with the increasing interest in p-EVs, it has been understood that pEVs are involved in hemostasis, vascular integrity, immunoregulation and inflammatory regulation and have various functions. In particular, different approaches have been evaluated in cases of injury and trauma, and it has also been observed that it has restorative effects on the musculoskeletal system and neural environment. It is considered that p-EVs may overcome the limitations of platelet concentrate and may even provide some desirable advantages that may increase the benefits of their clinical use. For example, pEVs not only share platelet function, but are also stronger in terms of coagulation or osteogenic capacity. When beyond hemostasis is considered, the content of pEVs is incredibly diverse and can include lipids, proteins, nucleic acids, and organelles involved in many other biological processes. In addition, while platelets cannot cross tissue barriers, their EVs can enter lymph, bone marrow, and synovial fluid and, similar to the other types of EVs, they are expected to be able to cross other tissue barriers, including the blood-brain barrier. This permits the transfer of platelet-derived content to cellular receptors and organs that cannot be accessed by platelets.

One of the major application areas of pEVs, which provides an important potential in regenerative medicine treatments, is injuries and wounds. The wound healing process is related to an increase in fibroblast and keratinocyte cell migration and proliferation in vitro. These effects are thought to be related to the cargo of pEVs positive for different growth factors, including platelet-derived growth factor (PDGF), basic fibroblast growth factors (FGF2), transforming growth factor- (TGF-), and vascular endothelial growth factor (VEGF). A study in a diabetic rat model confirms that pEVs can improve proliferation, migration and angiogenesis of vascular endothelial cells through activation of Yes-associated protein (YAP) and thereby confirms that it promotes wound healing in vivo. In addition to their wound healing properties, pEVs are also involved in the inflammatory response. Studies report that pEVs offer an anti-inflammatory effect on stimulated macrophages, reducing the release of cytokines such as tumor necrosis factor alpha (TNF-a) or interleukin 10 (IL-10). In addition to these, pEVs have been reported to be involved in neuroregenerative response and musculoskeletal regeneration. Studies of pEV-treated chondrocyte have shown that pEVs cause an increase in proliferation and cell migration through activation of the Wnt/-catenin signaling pathway, also a decrease in the proinflammatory response and the rate of apoptosis induced by inflammatory conditions. When the abovementioned pre-clinical study outcomes are taken into consideration, it is possible to say that PRP-EV-based therapies are promising for future tissue regeneration clinical applications.

The platelet activation process is closely related to the release of platelet-derived exosomes. Chemical activation methods have been broadly used as the activation method in the literature. There are various mechanical activation methods such as sonication and centrifugation as an alternative to chemical methods. Platelet activation caused by different activation factors (physical/chemical) leads to the release of pEVs that differ in size and content, but also increases exosome release. The vesicles released by the platelets under mechanical forces or in the presence of biochemical reagents such as thrombin differ in terms of their content and properties. For example, while activation of platelets by thrombin or collagen creates visible differences in terms of proteins in the content of exosomes, platelets activated by mechanical methods provide more exosome release to the external environment under the stress they are exposed to.

Today, there are new methods and new kits developed so as to isolate exosomes and EVs. Differential and ultracentrifugation, immunoaffinity, size elimination chromatography, ultrafiltration, and polymer-based precipitation are among the most widely used methods so as to obtain EVs and exosomes from animal and plant sources. However, each isolation method affects exosomes depending on the type of isolation according to density, form, scale and surface proteins. Meanwhile, since exosomes vary in function, size and content and are complex in structure, the probability of obtaining high purity fully isolated exosomes is low. In particular, the major disadvantage of ultracentrifugation method is that it does not isolate pure exosomes and the final exosome recovery is a bit low, also the process takes 2-3 days. It is difficult to use the polymer precipitation method clinically because there is a polymer removal phase therein. The major disadvantage of the size screening method is the long process time, which limits chromatographic isolation applications for processing a plurality of biological samples. Immune affinity isolation method is not applicable for large sample volumes. In addition, isolated vesicles may lose their functional activity.

Due to the abovementioned disadvantages and the insufficiency of the current products designs regarding the subject matter, a development is required to be made in the relevant technical field.

### SUMMARY

The present invention relates to an isolation kit which fulfils the abovementioned requirements, eliminates all disadvantages and brings some additional advantages.

The main aim of the present invention is to enable activation, filtration and concentration of a biological material in a closed environment with a single device. The device provides ease of use in an environment such as an operating room because it can operate in a closed environment and is portable.

Another indirect advantage of the present invention is that while obtaining a molecule of a certain size, the volume of the liquid is reduced, namely, its concentration is increased by filtering the molecules of undesired sizes and by filtering out the excess water. Filtration is typically carried out so as to separate, clarify, modify and/or concentrate a liquid solution, mixture or suspension. Filtration provides that undesired particle sizes and soluble molecules are separated from the exosomes. The exosome population is concentrated by the filter element during the process. Tangential-flow filtration (TFF) is a method that combines permeable membrane filtration and flow so as to obtain an efficient EV concentration from a colloidal matrix. TFF differs from traditional filtration since it flows tangentially along the liquid surface and prevents the formation of filter cake, thus allows exosomes to be obtained with high efficiency.

Another aim of the invention is to ensure that molecules in desired size ranges can be obtained by providing that the undesired size molecules and the water in the liquid can be evacuated.

The major aim of the present invention is to ensure that exosomes, nanoparticles or protein molecules with high activation capacity can be obtained especially for use in the production of nanoparticles without changing the medium from which biological fluids are taken, for example, without the need to transfer them to the laboratory.

Another aim of the present invention is to develop a kit that enables the transfer of exosomes/nanoparticles/proteins to be obtained, without contact with air, to the person, especially for use in obtaining nanoparticles of certain sizes, eliminating the risk of infection and contamination.

Another aim of the present invention is to develop a kit that allows the nanoparticles to be obtained with the help of the closed system to be transferred to the person without losing their stability and effectiveness.

Another aim of the present invention is to develop a kit wherein exosomes can be obtained in an activated form in a closed system, especially without loss of regenerative capacity.

Another aim of the present invention is to develop a kit wherein nanoparticles of certain sizes can be obtained based on mechanical methods without being exposed to any chemical or biological step or polymer.

Another aim of the present invention is to develop a kit that provides a closed environment with a filter system, eliminates undesired particles with a tangential flow filter cartridge, and increases the activation of glass beads and nanoparticles in the biological fluid, allows the delivery of the final product with a high regenerative capacity to the person.

Another aim of the present invention is to develop a kit with high efficiency, low cost and practical use for the extraction and concentration of exosome/nanoparticle/protein.

The structural and characteristic features of the present invention will be understood clearly by the following detailed description. Therefore the evaluation shall be made by taking this detailed description into consideration.

### BRIEF DESCRIPTION OF THE DRAWINGS

FIG. 1, is an exploded perspective view of the inventive isolation kit.

FIG. 2 is the cross-sectional view of the inventive isolation kit, taken parallel to the x-y plane.

FIG. 3, is the cross-sectional view of the inventive isolation kit, taken parallel to the y-z plane from the axis of the inlet port number 3,

FIG. 4, is the cross-sectional view of the inventive isolation kit, taken parallel to the y-z plane from the axis of the inlet port number 7,

FIG. 5, is the cross-sectional view of the inventive isolation kit, taken parallel to the x-y plane from the axis of the inlet port number 7,

FIG. 6, is a perspective view of the closed state of the inventive isolation kit,

FIG. 7, is a perspective view of the intermediate layer of the inventive isolation kit.

FIG. 8, is a bottom perspective view of the upper cover of the inventive isolation kit,

FIG. 9, is a top perspective view of the upper cover of the inventive isolation kit,

FIG. 10, is a perspective view of the activation chamber,

FIG. 11, is a perspective view of the filtration chamber,

FIG. 12, is a top perspective view of the lower cover of the inventive isolation kit,

FIG. 13, is a perspective view of the filter element,

FIG. 14, is a cross-sectional view of the filter element,

FIG. 15, is a bottom perspective view of the intermediate layer of the inventive isolation kit.

### PART REFERENCES

1. Inlet port number 1
2. Inlet port number 2
3. Inlet port number 3
4. Inlet port number 4
5. Inlet port number 5
6. Inlet port number 6
7. Inlet port number 7
8. Activation Chamber
9. Connection element number 1
10. Connection element number 2
11. Glass beads
12. Filter Element
13. Connection element number 3
14. Filtration Chamber
15. Tangential flow filter element
16. Connection element number 4
17. Connection element number 5
18. Isolation Kit
19. Tab
20. Stabilizing Feeder 1
21. Stabilizing Feeder 2
22. Lower Cover
23. Upper Cover
24. Tab Slot
25. Interlayer
26. Tab Gap
a. Removed from the inlet port number 2 and attached to the inlet port number 3.
b. Removed from the inlet port number 4 and attached to the inlet port number 5.

### DETAILED DESCRIPTION OF THE EMBODIMENTS

In this detailed description, the inventive isolation kit (18) is described only for clarifying the subject matter in a manner such that no limiting effect is created.

In particular, the inventive isolation kit (18) has been developed so as to obtain pure exosomes, nanoparticles or proteins. There are 3 different stages in product design.
1. Activation
2. Dead-end filtration
3. Tangential flow filtration

It relates to a product suitable for single or repeated use, which is based on glass beads for activation, dead end filtration and tangential-flow filtration units so as to remove undesired cellular debris, which can perform the filtration process with a single device with high efficiency without disturbing the closed system conditions.

Obtaining a concentrated and ready to apply exosome, nanoparticle or protein that has been activated, purified from undesired molecules, using the inventive kit, briefly, the operating principle of the inventive isolation kit is as follows;

Source material is obtained from a variety of sources, including cell culture, biological material, plant, blood plasma. The source material obtained is filled into a syringe with a Luer Lock tip. The syringe filled with source material is connected to the inlet port number 1 (1) located on the upper surface of the isolation kit (18). Another sterile, hollow syringe with Luer Lock inlet is connected to the inlet port number 2 (2) on the upper surface of the isolation kit (18). The source material is transferred approximately 15-20 times, first from 1 to 2, and then from 2 to 1, by syringe plungers, between the syringe at the inlet port number 1 (1) and the syringe at the inlet port number 2 (2). This transfer of the source material between the syringes is provided through the activation chamber (8) filled with glass beads (11). The connection of the inlet port number 1 (1) to the activation chamber (8) is provided by the connection element number 1 (9), the connection of the inlet port number 2 (2) to the activation chamber (8) is provided by the connection element number 2 (10).

The source material is exposed to high flow stress with the help of the medical grade glass beads (11) located in the activation chamber (8), thus, it is aimed to increase exosome or protein releases by increasing the activation of the source material by mechanical means by means of the medical grade glass beads (11). Undesired materials, cell wastes or high-dimensional proteins are also present in the liquid during this activation.

The material from the activation process is collected in the syringe connected to the inlet port number 2 (2). At this stage, there may be undesired or waste molecules in the obtained activated material. Here, the syringe, which is attached to the inlet port number 2 (2) and filled with the activated material, is “removed from the inlet port number 2 (2) and attached to the inlet port number 3 (3)” (a) so as to filter out these undesired molecules, another sterile and empty syringe is attached to the inlet port number 4 (4). It is ensured that all the material in the syringe passes into the sterile and empty syringe, which we connect to the inlet port number 4 (4) by depressing the syringe plunger, which is activated and filled with undesired/waste molecules, which we remove from the inlet port number 2 (2) and attach the same to the inlet port number 3 (3). The connection between the inlet port number 3 (3) and the inlet port number 4 (4) is provided by the filter element (12) and the connection element number 3 (13). Activated and filtered material, that is, material purified from cell wastes or high-dimensional proteins, is obtained in the syringe that we connect to the inlet port number 4 (4) as a result of this process.

As a final step, the tangential flow filtering element (15) in the filtration chamber (14) locates inside the device is used so as to obtain concentrated exosomes, nanoparticles or proteins from the activated and filtered material, that is, so as to remove the liquid inside the material. The filtration principle known as tangential flow filtration (cross flow filtration or tangential flow filtration/TFF) is used in said filtration chamber (14).

From the syringe, which is connected to the inlet port number 3 (3) in the previous step and contains the activated material, to the syringe, which is filtered and transferred to the empty syringe connected to the inlet port number 4 (4), by means of the filter element (12), and thus it is filled with the activated and filtered material, is removed from the inlet port number 4 (4) and attached to the inlet port number 5 (5)″ (b). Empty sterile syringes are connected to inlet port number 6 (6) and inlet port number 7 (7). The connection of the inlet port number 5 (5) to the filtration chamber (14) is provided by the connection element number 4 (16), and the connection of the inlet port number 6 (6) to the filtration chamber (14) is provided by the connection element number 5 (17). Transfer is made between the syringe with the activated and filtered source material removed from the inlet port number 4 (4), inserted into the inlet port number 5 (5) and the empty syringe attached to the inlet port number 6 (6) until the desired volume of exosome, nanoparticle or protein molecule is obtained. The liquid (water) and small molecules in the material exit from the inlet port number 7 (7) during this transfer. The aim of this process is to concentrate the activated and filtered molecules. The liquid containing the molecules is filtered by means of the tangential flow filter cartridge during the concentration, the selected very small molecules and the liquid are filtered with this filtration and the material is concentrated. For this reason, while transfer is made between the syringe attached to the inlet port 5 (5) and the syringe attached to the inlet port 6 (6), these molecules, which we can call undesired or waste and whose molecular dimensions are smaller than the exosome, nanoparticle or protein molecules we want to obtain, are filtered by the tangential flow filter element (15) and filled into the syringe connected to the inlet port number 7 (7). The material that fills the syringe connected to the inlet port number 7 (7) is the undesired waste material. Exosomes, nanoparticles or proteins ready for administration are collected and the syringe is removed from the isolation kit for administration in the syringe connected to port number 6 (6).

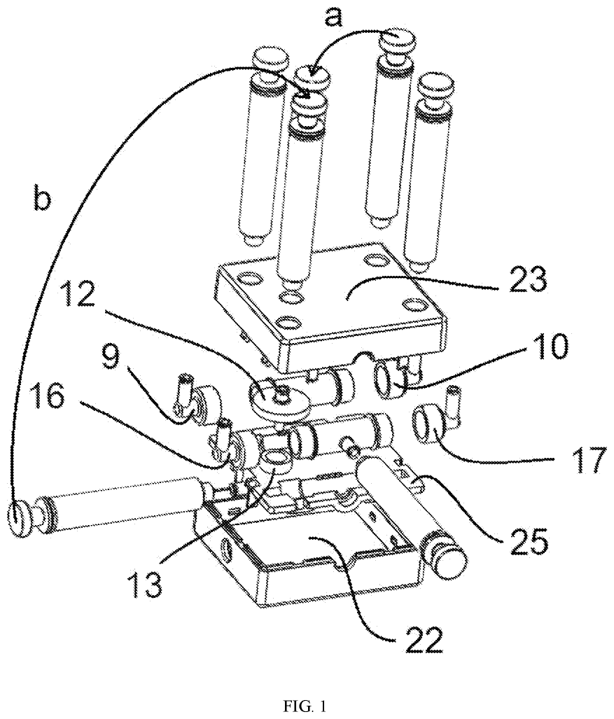

The exploded perspective view of the inventive isolation kit (18) is illustrated in FIG. 1. Although a total of 7 syringes can be seen schematically in this figure, the syringe attached to the inlet port number 2 (2) as described above and shown as a and b in the figure, is taken from here and attached to the inlet port number 3 (3). In addition, the material transferred to the inlet port number 4 (4) by being filtered at once from the syringe attached to the inlet port number 3 (3) is filled into the syringe here. The syringe attached to the inlet port number 4 is removed from here and attached to the inlet port number 5 for the next tangential-filtration operation. In summary, a total of 5 different syringes are used during these processes.

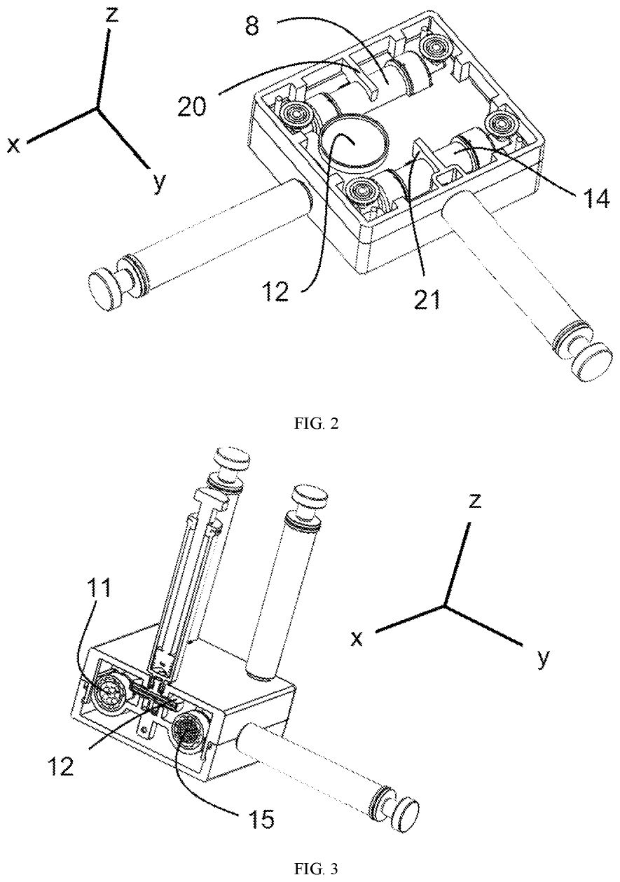

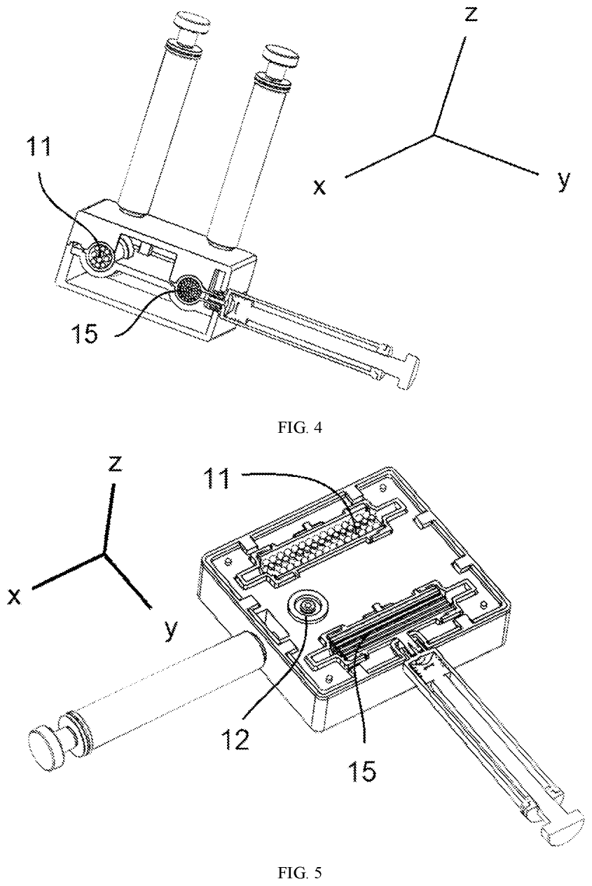

FIG. 2 shows the cross-sectional view of the inventive isolation kit, taken parallel to the x-y plane just below the upper cover. In this drawing, stabilizer feeder 1 (20) and stabilizer feeder 2 (21) located on the upper cover (23) and which provides bedding to the activation chamber (8) and the filtration chamber from the top when the upper cover (23) is closed are seen.

The cross-sectional view of the inventive isolation kit, taken parallel to the y-z plane from the inlet port number 3 (3) axis is illustrated in FIG. 3. The glass beads (11) in the activation chamber (8), the filter element (12) and the tangential-flow filter element (15) in the filtration chamber (14) can be seen in this drawing.

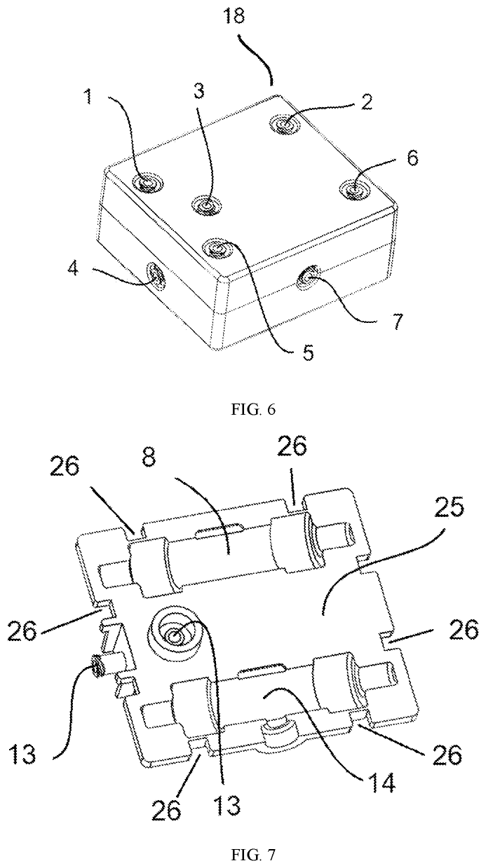

The perspective view of the closed state of the inventive isolation kit is illustrated in FIG. 6. A total of 7 syringe inlets on the isolation kit can be seen in this drawing.

A perspective view of the intermediate layer (25) of the inventive isolation kit (18) is illustrated in FIG. 7. The intermediate layer (25) positioned between the lower cover (22) and the upper cover (23) provides bedding to the activation chamber (8) and the filtration chamber (14). The connection element number 3 (13) is also positioned on said intermediate layer (25). Moreover, tab gaps are formed on said intermediate layer (25) in accordance with the positions of the tabs (19) so that the tabs (19) positioned circumferentially on the upper cover can reach the tab slots (24) on the lower cover.

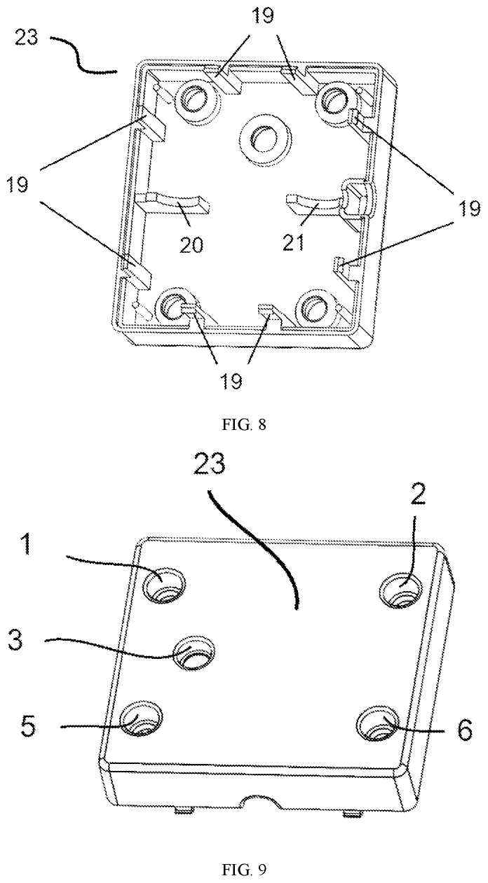

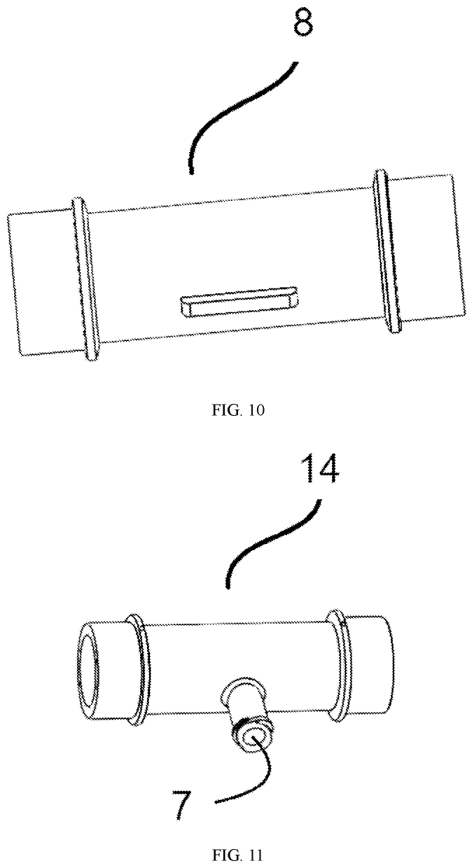

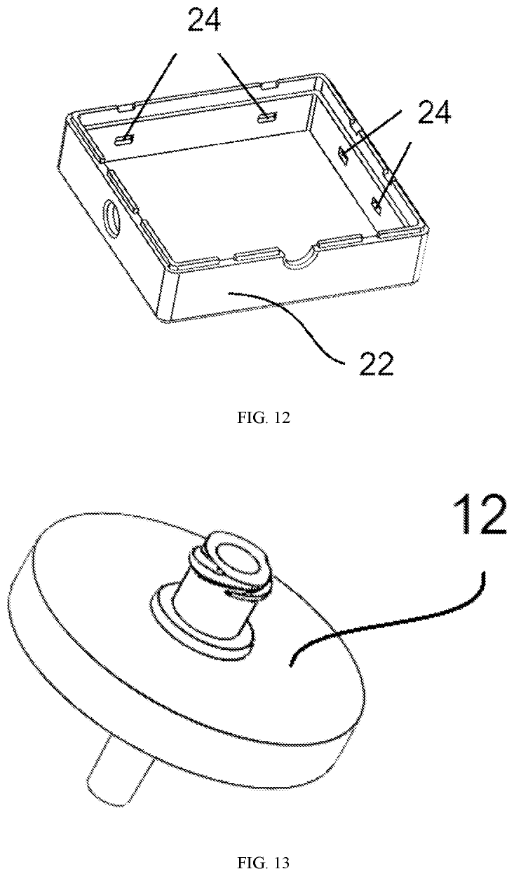

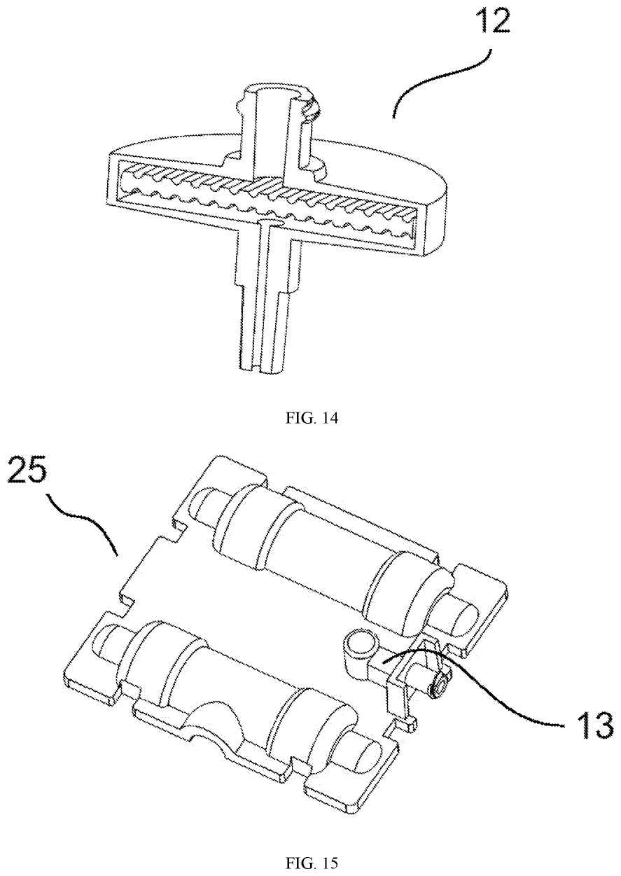

The tabs (19) on the upper cover (23) of the inventive isolation kit (18) can be seen in FIG. 8. The assembly of the lower cover (22) and the upper cover (23) to each other is achieved by engaging the tabs (19) on the upper cover (23) into the tab slots (24) on the lower cover (22).

What is claimed is:

1. A portable and combined isolation kit that will enable obtaining activated exosome molecules, nanoparticles or protein molecules in certain sizes from various sources including cell culture, biological material, plant, blood plasma, wherein the isolation kit comprises the following:
an activation chamber with glass beads inside so as to enable an activation of exosomes, nanoparticles or protein molecules,
a filter element so as to filter molecules larger than the desired molecular size in an activated material,
a filtration chamber with a tangential-flow filtering element inside so as to increase a filtration and concentration of undesirably small molecules and liquid in the filtered material with large molecules,
an inlet port number 1 so as to connect a syringe containing a material to be activated to the isolation kit,
an inlet port number 2 wherein the inlet port number 2 is connected to a syringe which the material will fill when pressed from the syringe connected to the inlet port number 1 and passes through the activation chamber with the glass beads,
an inlet port number 3 to wherein the inlet port number 3 is connected to the syringe connected to the inlet port number 2, which is filled with activated material inside after 15-20 transfers between the syringe connected to the inlet port number 1 and the syringe connected to the inlet port number 2, is connected for filtering, after being removed from the inlet port number 2,
an inlet port number 4, wherein the inlet port number 4 is connected to a syringe which the molecules larger than desired and in the material pressed from the syringe that is removed from the inlet port number 2 and connected to the inlet port number 3 will be filled after being filtered by the filter element,
an inlet port number 5, where the syringe in the inlet port number 4 will be removed and attached, so as to filter the molecules found in the material activated by repeated passing through glass beads, and whose molecules larger than desired are filtered by passing though the filter element and to filter out undesirably small molecules,
an inlet port number 6 so as to connect a syringe into which molecules of desired sizes will be filled while the material pressed from the syringe attached to the inlet port number 5 after being removed from the inlet port number 4, passes through the tangential flow filtering element in the filtration tube,
an inlet port number 7 so as to connect a syringe into which molecules and liquid of smaller sizes than desired will be filled while the material pressed from the syringe attached to the inlet port number 5, passes through the tangential flow filtering element in the filtration tube.

2. The isolation kit according to claim 1, further comprising a connection element number 1 so as to ensure the connection of the inlet port number 1 to the activation chamber.

3. The isolation kit according to claim 1, further comprising connection element number 2 so as to ensure the connection of the inlet port number 2 to the activation chamber.

4. The isolation kit according to claim 1, further comprising a connecting element number 3 positioned at the bottom of the filter element so as to connect the inlet port number 3 with the inlet port number 4.

5. The isolation kit according to claim 1, further comprising a connection element number 4 so as to ensure the connection of the inlet port number 5 with the filtration chamber.

6. The isolation kit according to claim 1, further comprising a connection element number 5 so as to ensure the connection of the inlet port number 6 to the filtration chamber.

7. The isolation kit according to claim 1, further comprising an upper cover.

8. The isolation kit according to claim 7, wherein the upper cover further comprises a circumferentially positioned tabs.

9. The isolation kit according to claim 7, wherein the upper cover further comprises a stabilizing feeder 1 that prevents the activation chamber from moving by pressing from the top when said upper cover is closed on the lower cover.

10. The isolation kit according to claim 7, wherein the upper cover further comprises a stabilizing feeder 2 that prevents the filtration chamber from moving by pressing from the top when said upper cover is closed on the lower cover.

11. The isolation kit according to claim 1, by further comprising a lower cover.

12. The isolation kit according to claim 11, wherein the lower cover further comprises tab slots where the tabs on the upper cover can be inserted.

13. The isolation kit according to claim 1, by further comprising an intermediate layer having slots on which the activation chamber and the filtration chamber will be located.

14. The isolation kit according to claim 13, wherein the intermediate layer further comprises tab gaps so that the tabs on the upper cover can fit into the tab slots on the lower cover.
